(19) United States
(12) Patent Application Publication
(10) Pub. No.: US 2023/0136992 A1
(43) Pub. Date: May 4, 2023

(54) METHOD FOR BLOOD PRESSURE MEASUREMENT

(71) Applicant: BIOBEAT TECHNOLOGIES LTD., Petach Tikva (IL)

(72) Inventors: JOHANAN MAY, PETACH TIKVA (IL); ISRAEL SARUSSI, GANEI TAL (IL); ARIK BEN ISHAY, ZORAN (IL)

(21) Appl. No.: 17/512,701

(22) Filed: Oct. 28, 2021

(51) Int. Cl. A61B 5/021; A61B 5/00; A61B 5/11; A61B 5/0205; A61B 5/024; A61B 5/08; A61B 5/145

(52) U.S. Cl. CPC A61B 5/021; A61B 5/746; A61B 5/4561; A61B 5/1113; A61B 5/0205; A61B 5/02405; A61B 5/0816; A61B 5/14542; A61B 2560/0252; A61B 2562/029

(57) ABSTRACT

A method for evaluating a normal range of a blood pressure, the method including executing a function, where the function receiving as input measured values of one or more physiological parameters of a person, where the function receiving as input measured values of one or more environmental parameters, and where the function outputs a normal range of blood pressure values for the person according to the measured values of one or more physiological and/or environmental parameters.

### FIELD AND BACKGROUND

The invention, in some embodiments thereof, relates to blood pressure (BP) measurement and, more particularly, but not exclusively, to a method for measuring blood pressure using multiple sensors.

Measuring BP values can be done using portable devices, for example devices in which the user pumps air into an inflatable balloon (referred to also as a BP cuff) until the balloon is sufficiently full in order to measure the patient's BP. Other BP measurements require electrodes coupled to the patient's skin, for example as part of a wearable device.

BP measurements and their significance vary from one person to another. For example, certain BP values may be considered normal for one person and be considered problematic for another person. When measuring BP during long periods of time, for example a month, BP measurements vary, for example when the user sleeps, works, exercises, when the user is under certain biological conditions and the like. These variations may be considered outside the normal range for a specific patient, although they result from different conditions the patient experiences.

Hence, there is a need to distinguish between BP variations caused by normal conditions the patient experiences and BP variations caused by abnormal medical conditions the patient suffers from, that should be addressed by a health care provider.

### SUMMARY

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In one aspect of the invention a method is provided for evaluating a normal range of a blood pressure, the method including executing a function, where the function receiving as input measured values of one or more physiological parameters of a person, where the function receiving as input measured values of one or more environmental parameters, where the function outputs a normal range of blood pressure values for the person according to the measured values of one or more physiological and/or environmental parameters.

In some cases, the method further includes measuring reference blood pressure values at several initial time points from the person, measuring additional reference values of one or more additional physiological or environmental parameters of the patient during the same several initial time points, where the one or more additional or environmental physiological parameters are not blood pressure, generating the function based on the measured reference blood pressure values and the reference values of the one or more additional physiological or environmental parameters.

In some cases, the normal range is defined between a minimal normal BP value and a maximal normal BP value, where minimal normal BP value and the maximal normal BP value depend on the one or more additional physiological or environmental parameters.

In some cases, the method further includes measuring test blood pressure value. In some cases, the method further includes measuring value of the one or more additional physiological or environmental parameters at the same time of measuring the test blood pressure value.

In some cases, the method further includes determining whether the measured test blood pressure value is within the normal range of the function pending on the measured additional parameter.

In some cases, the method further includes outputting a notice or an alert in case the measured test blood pressure value of the patient differs from the normal range of values. In some cases, the method further includes outputting a notice or an alert in case the measured test blood pressure value differs from the normal range by a value higher than a predefined threshold. In some cases, the test blood pressure value is measured in a continuous manner by a measuring device coupled to a patient.

In some cases, the method further includes updating the normal range according to one or more environmental conditions or activities. In some cases, the one or more environmental conditions are selected from a group including hospitalization, patient's velocity relative to the ground, patient's location, patient's body posture, ambient conditions like temperature, humidity, height above sea level and a combination thereof. In some cases, the method further includes updating the function for blood pressure values using additional blood pressure values measured after initial several time points. In some cases, the one or more additional physiological or environmental parameters include at least one of a group including heart rate, heart rate variability, breathing rate, blood oxygen saturation, and a combination thereof.

In some cases, the reference blood pressure values include diastolic blood pressure values and systolic blood pressure values. In some cases, measuring reference values of two or more additional physiological or environmental parameters other than the blood pressure during the initial several times points, and generating the function based on the two or more additional physiological or environmental parameters.

In some cases, the function is a lookup table associating the reference blood pressure values and the one or more additional physiological or environmental parameters values. In some cases, measuring the reference blood pressure values is during a predefined baseline time period. In some cases, generating the function is by one or more techniques selected from a group including polynomic fit, interpolation and extrapolation. In some cases, the method further includes calculating an adjusted BP value using the measured BP value and the reference values of the one or more additional physiological or environmental parameters.

In some cases, measuring of the test blood pressure value is performed in a periodical manner. In some cases, the reference blood pressure values include at least one of systolic or diastolic BP values. In some cases, the test blood pressure value includes at least one of systolic or diastolic BP values. In some cases, the personalized BP function is generated according to rules that consider objective information of the person. In some cases, the personalized BP function is generated according to additional reference values. In some cases, the normal range is personalized. In some cases, the normal range is generated according to rules that consider objective information of the person. In some cases, the normal range is generated according to additional reference values of the person.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A and 1B are flow diagrams of some examples of processes for measuring blood pressure, according to an embodiment of the invention;

FIG. 1C shows a method for evaluating a normal range of a blood pressure, according to an embodiment of the invention;

FIG. 2 is a table of an example list of additional physiological reference parameters and potential BP function, according to an embodiment of the invention;

FIG. 3 shows a system for measuring and evaluating blood pressure, according to an embodiment of the invention;

FIG. 4 shows a schematic illustrations of a measuring device for measuring clinical parameters of a patient, according to an embodiment of the invention.

### DETAILED DESCRIPTION

The invention, in some embodiments thereof, relates to blood pressure measurement and, more particularly, but not exclusively, to a method for noninvasive measurement of blood pressure.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

According to an aspect of the invention, there is provided a method for evaluating blood pressure measurements. In some examples, the method includes measuring reference blood pressure values of a patient at several initial time points. The method includes measuring additional reference values of one or more additional physiological parameters of the patient or other environmental parameters during the same several initial time points, both defined as additional parameters. The one or more additional physiological parameters are not blood pressure. The method then includes generating a BP function for blood pressure values that depend on the additional reference values of the one or more additional physiological parameters. In some embodiments, the initial time points may be defined prior to measuring the reference blood pressure values. In some embodiments, the initial time points are defined in accordance with a quality determination of the generated BP function. For example, initial time points may be added if the BP function does not converge. In another example, initial time points may be reduced after the function converges.

According to an aspect of the invention, there is provided a measurement device. The measurement device measures baseline values of both diastolic and systolic blood pressure (BP) and optionally other physiological parameters during a baseline period.

In some example configurations, the device and method are adapted to be used at home, office, in a vehicle, in a clinic, or hospital or other specific environments. In some example configurations, the device and method are adapted to be operated by a non-professional person, a patient, a technician, or a healthcare professional.

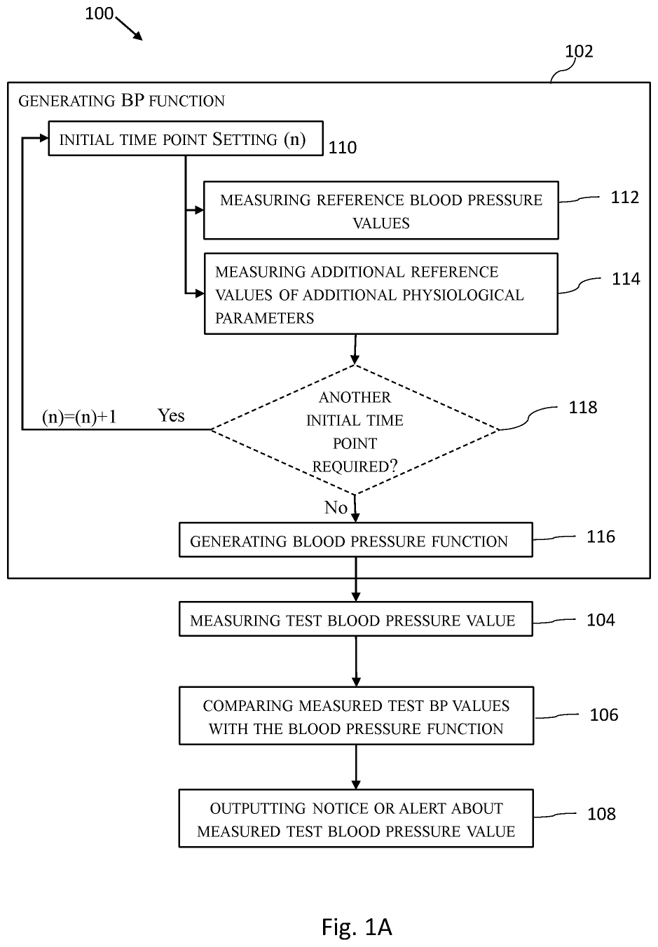

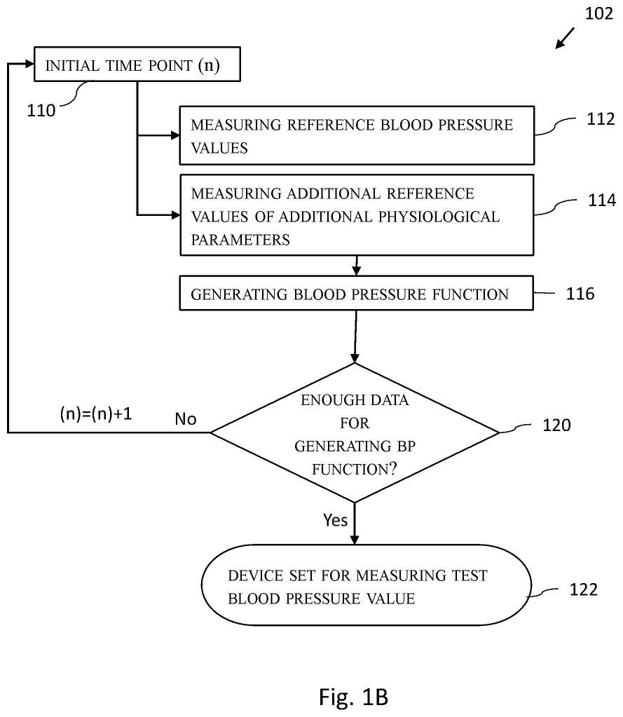

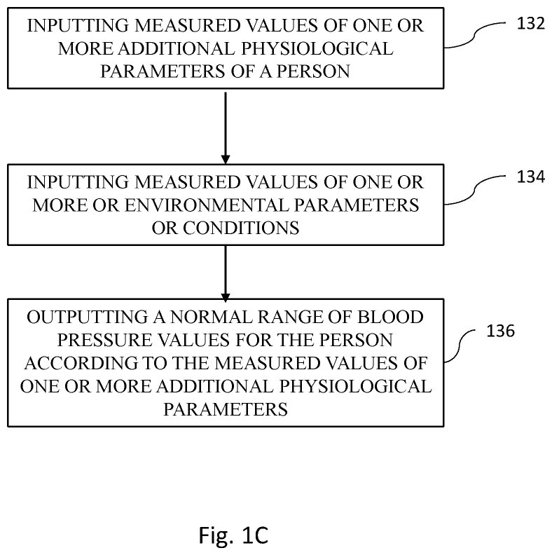

Reference is now made to FIGS. 1A to 1B which are flow diagrams of some examples of processes for measuring and evaluating blood pressure values, according to an embodiment of the invention. As shown in FIG. 1A, process 100 is used for measuring and evaluating blood pressure values.

Step 102 discloses generating a BP function for blood pressure values that are generated using reference measured blood pressure values and additional reference measured values of one or more additional physiological parameters. The BP function enables to evaluate the BP values of a specific patient based on measurements of one or more additional physiological parameters. For example, when the BP values are high than a normal range, and the one or more additional physiological parameters are also high, the BP function will assist in considering that the high BP value is normal as it correlates with the increase in the measurement of the one or more additional physiological parameters.

Step 104 discloses measuring test blood pressure together with the additional reference physiological parameter/s value/s. The test BP and reference physiological parameter/s values may be measured continuously, periodically, intermittently, at time schedule and spot check, for example once every set number of 2.5 seconds, minutes or hours during a set period of time. The set period of time for measuring the test blood pressure values may be for example 1 to 14 days. Alternatively, the measurement can be done periodically, every day or every number of days for a set period (typically 1-12 months). The test BP values may be measured using various BP devices such as a wearable device coupled to the patient's body, for example using adhesives.

Step 106 discloses comparing measured test BP values with the generated BP function using the reference physiological parameter/s value/s. The function may provide a normal BP value for the given additional physiological parameter/s value, or provide a range of BP values considered as normal range. For example, in case the one or more additional physiological parameters include heart rate, the function dictates that heart rate of 70 beats per minute matches a systolic BP value of 120 mmHg with range of 115 to 125 mmHg. Similarly, the function may dictate that heart rate of 90 beats per minute matches a systolic BP value of 140 mmHg with range of 130 to 150 mmHg. This way, when the test BP systolic value is 140 mmHg, the test value is considered normal if the heart rate is 90 beats per minute and considered out of the normal range in case heart rate is 70 beats per minute. Typically, when heart rate is higher, the function will provide higher BP values as normal values.

According to some embodiments, process 100 includes step 108 of outputting a notice or an alert about the measured test value of BP. In some embodiments, the notice includes the values of the BP. In some embodiments, the notice includes an alert. For example, an alert is generated in case the blood pressure test value differs from the normal range of values as in the BP function. In some embodiments, outputting notice 108 is in case the BP test value differs from the normal range by a value higher than a predefined threshold.

According to some embodiments, the step 102 of generating a BP function includes one or more of the following steps:

Step 110 discloses setting initial time points. According to some embodiments, the initial time points are defined prior to the step 102 of generating a BP function. The initial time points define the time points used to measure the reference blood pressure values, for example 10 times per second during two weeks. Alternatively, generating the BP function may continue until the system stored a sufficient number of measured values, or a sufficient variance in values of the reference physiological parameter/s. For example, having a minimal number of measurements events (for example 100 events) having variance of at least set value (for example 10%).

According to some embodiments, the period in which the initial time points are set is between 1 day and 1 month.

Step 112 discloses measuring reference blood pressure values at several initial time points. In some embodiments, measuring the reference blood pressure values is performed during a predefined baseline time period.

Step 114 discloses measuring additional reference values of one or more additional physiological parameters during the same several initial time points. The one or more additional physiological parameters are not blood pressure. Measuring the additional reference values of one or more additional physiological parameters during the same several initial time points used to measure the reference blood pressure values enables to associate a wide range of BP measurements to measurements of the one or more additional physiological parameters measured during substantially the same time of the BP measurement. The term “substantially same time” is defined as a time offset short enough to indicate that the patient was under the same physiological conditions when measuring the BP and when measuring the one or more additional physiological parameters.

According to some embodiments, measuring steps 112 and/or 114 are performed for a set period of time. In some embodiments, the set period of time is between a few seconds to 24 hours. In some embodiments, measuring steps 112 and/or 114 may repeat for between 1 to 50 measurement sections.

Step 116 discloses generating one or more BP functions for blood pressure values that depend on the additional reference values of the one or more additional physiological parameters. In some cases, the one or more BP functions may include a continuous function, for example using interpolation and extrapolation values.

The BP function may vary from one patient to another, for example according to objective information such as age, gender, and medical conditions, or according to measurements taken during the initial time points. Similarly, the normal range of BP values may also be computed specifically for each patient, or a group of patients, the group may be defined by gender and age. For example, suppose every value of the additional parameter measurements have a BP value for a specific person. The normal range of BP values for the specific person may be determined for the specific user. For example, for one user the normal range may include a deviation of 10% from the BP value, while for another person the normal range may include a deviation of 15% from the BP value.

Optionally, the step 102 of generating a BP function includes the step 118 of checking if other initial time points are required to define the BP function. In some embodiments, if other initial time points are required the process 102 of generating a BP function includes adding initial time points, e.g., by repeating one or more of steps 110 to 114. If the BP function is successfully generated, the measuring device is set for testing BP values.

FIG. 1B, shows an alternative example of a step of generating a BP function in which the step of generating one or more BP functions is followed by step 120 of evaluating the quality of the generated BP function by checking if there is enough data for generating the BP function. In some embodiments, if there is not enough data, the process 102 of generating a BP function includes adding initial time points, e.g., by repeating one or more of steps 110 to 116. If the BP function is successfully generated, the measuring device is set for measuring the test BP values in step 122.

According to some embodiments, step 116 of generating BP function includes defining a normal range of blood pressure values as a function of the one or more additional physiological parameters. In some embodiments, the normal range is defined between a minimal normal BP value and a maximal normal BP value, where minimal normal BP value and the maximal normal BP value depend on the one or more additional parameters. In some embodiments, step 116 of generating BP function includes defining a baseline value of the diastolic blood pressure. In some embodiments, step 116 of generating BP function includes defining a baseline value of the diastolic blood pressure. In some embodiments, the BP function is a lookup table associating the reference blood pressure values and the one or more additional parameters values. In some cases, step 116 of generating BP function is performed using one or more techniques, for example selected from a group including polynomic fit, interpolation, and extrapolation.

According to some embodiments, step 104 of measuring test BP value includes measuring one or more additional physiological parameters at the same time of the test blood pressure value measurement. The one or more additional physiological parameters may be of the additional physiological parameters that defines the BP function, such as additional reference values of additional physiological parameters measured by sensors used in step 116. In some embodiments, measuring 104 test BP value is in a continuous manner.

According to some embodiments, step 106 of comparing test BP pressure values with the generated BP function includes determining whether or not the measured test blood pressure value of step 104 is within the normal range of blood pressure.

According to some embodiments, process 100 includes updating the generated BP function and the BP baseline values. Updating may be performed using a new measurement event. In some embodiments, process 100 includes updating the generated BP function using additional blood pressure values measured after the initial several time points measured in step 104. In some embodiments, updating the BP function or a normal range is performed according to one or more environmental conditions or activities. Some examples of such environmental conditions or activities may include hospitalization, patient's velocity relative to the ground (measured for example by an accelerometer of the measurement device coupled to a patient's organ), patient's location (measured for example by a location detection unit of the measurement device), patient's body posture (identified by the measuring device or as an additional input of an operator), ambient conditions like temperature, humidity, height above sea level time of day and a combination thereof.

FIG. 1C shows a method for evaluating a normal range of a blood pressure, according to an embodiment of the invention.

Step 132 discloses inputting measured values of one or more additional physiological parameters of a person. The inputting may be provided automatically from a device that measures the one or more additional physiological parameters of the person to the device that performs the process of evaluating a normal range of a blood pressure.

Step 134 discloses inputting measured values of one or more or environmental parameters or conditions. The environmental parameters Some examples of such environmental conditions or activities may include hospitalization, patient's velocity relative to the ground (measured for example by an accelerometer of the measurement device coupled to a patient's organ), patient's location (measured for example by a location detection unit of the measurement device), patient's body posture (identified by the measuring device or as an additional input of an operator), ambient conditions like temperature, humidity, height above sea level time of day and a combination thereof.

The function's formula or rules may be stored in a memory device accessible to the device into which the measured values are inputted. In some cases, the function's values are determined by an operator, for example the patient, the patient's representatives, a medical doctor, a nurse, a technician, or other medical-related personnel.

Step 136 discloses outputting a normal range of blood pressure values for the person according to the measured values of one or more additional physiological parameters. This way, a person, either the patient or a medical person treating the patient, can evaluate whether or not the patient's blood pressure measurements are normal, or whether the person should be alerted.

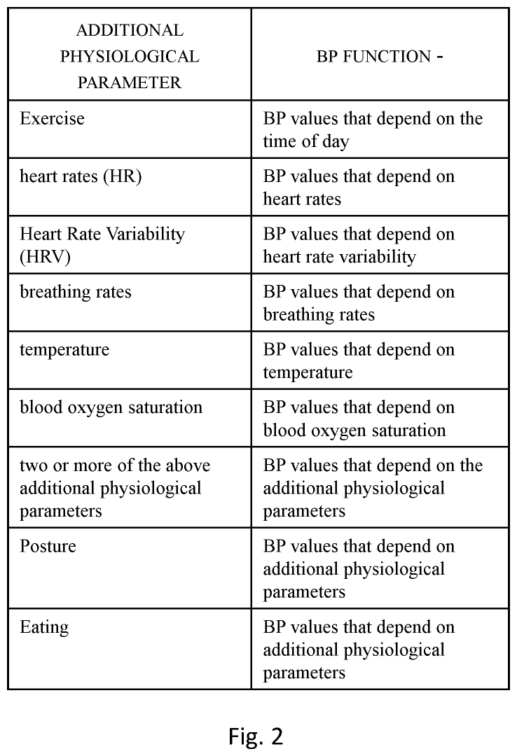

Turning to FIG. 2, which shows a table of an example list of additional physiological reference parameters and potential BP function, according to an embodiment of the invention.

Based on the BP measurements and one or more parameter measurements that may include the time of day or one or more physiological parameters' measurements, the measuring device may compute baseline values for BP. The computation can be a function of the measured values on the BP and the one or more parameters. The device may use polynomic fit or interpolation of values between measured values to provide complete range of BP baseline values that depend on the one or more physiological parameters. The BP baseline values function can take into consideration several different measurements taken at different measurement events (e.g., initial time points 1−n). An example of such a consideration is for example averaging between values of BP baseline values calculated or measured at the different measurement events.

As shown in the table of FIG. 2, the one or more additional physiological parameters may include at least one of a group including heart rate, heart rate variability, breathing rate, blood oxygen saturation, temperature, and a combination thereof. In some embodiments, the BP function depends on reference values of two or more additional physiological parameters other than the blood pressure during the initial times points and generating the BP function is associated with the measured reference values of the two or more additional physiological parameters.

In some embodiments, the BP function may depend on one or more of measured diastolic blood pressure values and systolic blood pressure values.

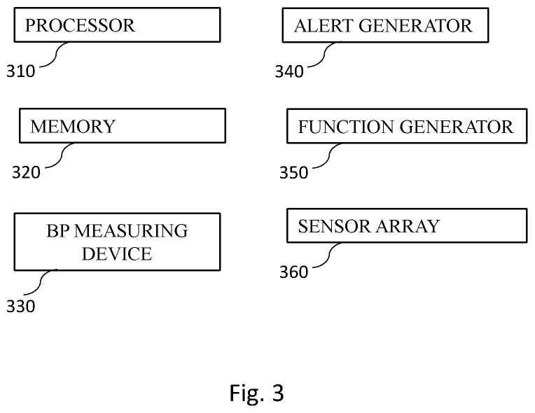

FIG. 3 shows a system for measuring and evaluating blood pressure, according to an embodiment of the invention.

The system includes processor 310. According to some embodiments, the processor 310 includes a processing functionality to generate one or more BP functions for blood pressure values. In some embodiments, the processor 310 includes one or more processing machines, for example processors, microprocessors, virtual machine processors and the like.

The system includes memory 320. The memory 320 may be used to store data such as data generated during the operation of the measuring device, for example sensors measurements. The memory addresses of the memory 320 may be accessible to the processor 310. The memory 320 may also store BP functions generated after measuring the reference BP values and the additional reference values of additional physiological parameters. The memory 320 may also be used to store data such as initial or updated settings of the measurement device, for example settings related to sensors, rules related to initial time points, time settings and the like.

The system includes BP measuring device 330 for measuring a patient's blood pressure values. According to some embodiments, the BP measuring device 330 includes a photoplethysmogram (PPG) sensor, including one or more light sources and one or more light detectors, used for continuous measurement of blood pressure. According to some embodiments, the measurement device further includes a data storage unit, and a processor.

In some example embodiments, the BP measuring device 330 includes one or more of the following sensors: (a) Electrogram (e.g., ECG/EEG/EMG/EOG/EGG) sensor for measuring body electrical activity of electrode-tissue impedance

The system includes alert generator 340. The alert generator 340 includes elements or components for alerting or displaying an abnormal condition, for example using audio, vibration, or visual indications. In some cases, the system includes components for communication with external devices, for example for sharing the BP measurements or to send the alerts.

The system includes function generator 350 configured for generating a BP function. The BP function defines the relation between measured BP values and values of other physiological parameters as measured during a reference period. The function generator 350 may be a software component operating in the system, implementing algorithms stored in the memory 320. The algorithms may be updated, for example by downloading content from the internet or from another device communicating with the system.

The system may include a sensor array 360 for collecting measurements other than blood pressure. The sensor array 360 may include one or more sensors, such as a temperature sensor for measuring temperature of one or more body regions. The regions include one or more of core, body, skin, near body ambient temperature. Some examples of temperature sensors are thermocouple, resistive temperature device or infrared sensors. Other sensors included in the sensor array 360 may be an accelerometer for measuring movement, vibration and rotations in all axes; and a GSR (galvanic skin response) sensor, for measuring the electrical conductance between 2 or more contact points on the patient's skin.

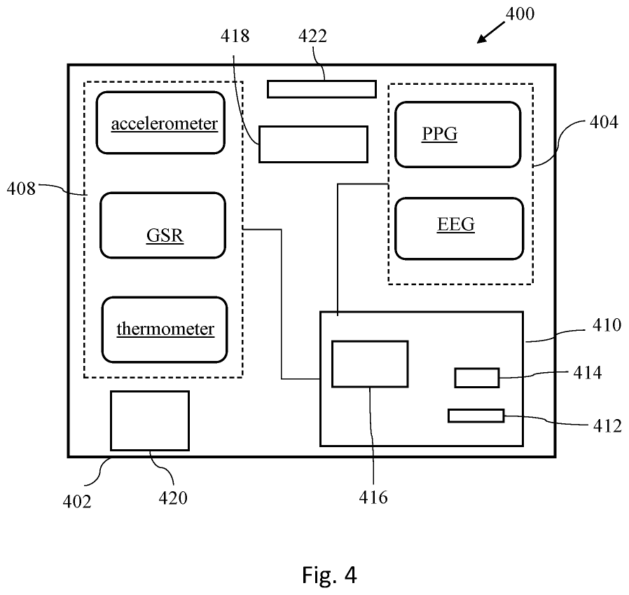

Referring now to the drawings. FIG. 4 shows schematic illustrations of a measuring device for measuring clinical parameters of a patient, according to an embodiment of the invention. Measuring device 400 has a housing 402, and two or more sensors located in the housing 402. According to some embodiments, the two or more sensors, can be sub-grouped into categories of sensors having shared characteristics, such as: type of measured parameters (e.g., electrical, mechanical, dynamics, optical), type of measured physical phenomenon (pressure, electrical conductivity, thermal, etc.), which clinical systems is being measured (e.g., cardiac, skin, muscular).

According to some exemplary embodiment, measuring device 400 has one or more cardiac or cardiovascular sensors 404 for outputting cardiac activity values. In some embodiments, cardiac or cardiovascular sensors 404 are selected from a photoplethysmogram (PPG) sensor and an electrogram sensor. In addition, measuring device 400 has one or more one or more additional sensors 408. In some embodiments, additional sensors 408 can be an arrangement of sensors selected to measure a variety of physical phenomenon such as a galvanic skin response (GSR) sensor, an accelerometer, and a thermometer.

According to some embodiments, measuring device 400 includes electrical circuitry 410 located in the housing 402 and electrically connected to the two or more sensors. In some embodiments, electrical circuitry 410 has a storage unit 412 for storing sensors data and sensors activation rules.

The sensors activation rules are defined to dictate which of the two or more sensors or sub-groups of sensors are used to sample the clinical parameters. In some example embodiments, electrical circuitry 410 is configured to activate one or more cardiac or cardiovascular sensors 404 when the measuring device 400 is set to measure clinical parameters. In some example embodiments, electrical circuitry 410 is configured to activate one or more additional sensors 408 when the measuring device 400 is set to measure clinical parameters. For example, the electrical circuitry may be configured to select a specific biological or medical parameter to be measured based on the biological or medical condition of the patient and to activate at least one of the two or more sensors in order to measure the selected specific biological or medical parameter. Some examples of the selected biological or medical condition are: Acute HF, COPD level, systemic infection (sepsis), pneumonia, sleep apnea, Hypertension, hypotension, fall, general deterioration, and a combination thereof.

In some embodiments, the measuring device 400 has at least five sensors, for example: a photoplethysmogram (PPG) sensor, an electrogram sensor, a galvanic skin response (GSR) sensor, an accelerometer and a thermometer. In some embodiments, the at least five sensors collect information in a continuous manner. The continuous manner may be defined as sampling measurements during a long period of time, for example at least one hour, one day, one week, one month and one year. In some embodiments, the at least five sensors collect information at set intervals. The set intervals may depend for example on a status of a battery providing electrical power to the measuring device 400.

According to some embodiments, electrical circuitry 410 has one or more processors 414 configured to process the sensors' data.

In some embodiments, electrical circuitry 410 includes a sensors switching circuit 416 configured to determine which sensors of the two or more sensors collect information in a given time frame in accordance with the sensors activation rules. The sensors switching circuit 416 may access data stored in the storage unit 412, such as the sensors activation rules.

According to some embodiments, electrical circuitry 410 is configured to measure a signal to noise ratio of at least one of the two or more sensors. In some embodiments, the sensors activation rules are used to select the two or more sensors in accordance with noise to ratio rate calculated based on preceding measurements received from the sensors. In some embodiments, the sensors activation rules include operational range, such as: measuring range, sensitivity, and a signal filtering profile. In some embodiments, the sensors activation rules are associated with a biological or medical condition of the patient. In some embodiments, the sensors activation rules are used to select the two or more sensors in accordance with an input that includes a selection of clinical parameters to measure or a measurement plan to define required measurements by the measuring device 400.

According to some embodiments, the sensors activation rules dictate that a first sensor of the two or more sensors is used to measure a specific biological or medical parameter when the measured values are within a first set range and a second sensor of the two or more sensors is used to measure the specific biological or medical parameter when the measured values are within a second set range. In some embodiments, at least two of the two or more sensors collect information simultaneously regarding the same medical parameter.

According to some embodiments, determining which sensors of the two or more sensors will measure the value of the clinical parameter in a given time frame is based on the patient's condition. In some embodiments, determining which sensors of the two or more sensors will measure the value of the clinical parameter in a given time frame is based on the signals measured from the two or more sensor.

According to some embodiments, one sensor of the two or more sensors is used to calibrate measurements of a second sensor of the two or more sensors.

According to some embodiments, the measuring device 400 has an input unit 420. In some embodiments, input unit 420 has an interface for a person to select operational functions or operational parameters. In some embodiments, input unit 420 is adapted for receiving the sensors activation rules. In some embodiments, input unit 420 includes a receiver for receiving data or operational parameters from a remote device.

In some embodiments, sensors data include sampled data received from the two or more sensors. In some embodiments, data is sampled in a rate which is adjusted in accordance with a quality score associated with the sampled data. In some embodiments, quality score of the sampled data and/or adjusting the rate is by a sampling functionality associated with processor 414.

The measuring device 400 may include an output unit 422 configured to receive signal values from the two or more sensors and to output clinical parameters. In some embodiments, signal values include sensors data processed by processor 414. In some embodiments, output unit 422 is configured to output other indications such as alerts based on ranges defined for one or more clinical parameters. In some embodiments, output unit 422 is configured to output indications about the quality of sensors data and/or signal values.

The measuring device 400 may include a storage unit 412, configured to store one or more of the following data fields: historical data received from the two or more of sensors, historical sensors activation instructions, and historical clinical parameters. In some embodiments, storage unit 412 stores a biological or medical condition of the patient, for example: one or more or a combination of Acute HF, COPD level, systemic infection (sepsis), pneumonia, sleep apnea, Hypertension, hypotension, fall, general deterioration. In some embodiments, storage unit 412 is used to store sensors data prior and/or after being processed by processor 414. In some embodiments, storage unit 412 is removable or allows to use a removable storage medium. In some embodiments, storage unit 412 is used to store the sensors activation rules. In some embodiments, storage unit 412 is used to store measurements plans used to define required measurements by the measuring device 400. In some embodiments, the sensors activation rules are adjusted to match a biological or medical condition of the patient. In some embodiments, storage unit 412 stores normal range of values of specific biological or medical parameters, and the electrical circuitry is configured to generate an alert in case one or more of the specific biological or medical parameters are outside the normal range of values.

In some embodiments, cardiac or cardiovascular sensors 404 have one or more sensing probes, such as a photoplethysmogram (PPG) sensor and an electrogram sensor. In some embodiments, photoplethysmogram (PPG) sensor has light sources and light detectors for detecting light reflections from a body tissue. The PPG sensor may control the transmitted light, for example by controlling the light spectrum and frequency. The PPG sensor may also analyze the returned signal by measuring the returned pulse shape and the tissue absorption. In some embodiments, light sources and/or light detectors are exposed to a body tissue via a tissue facing surface of housing 402.

In some embodiments, measuring device 400 includes one or more thermometers for measuring one or more body regions. The body regions may be, for example: core, body, skin, near body regions and the like. The thermometer may include one or more sensors such as thermocouple, resistive temperature device, and infrared sensors.

In some embodiments, measuring device 400 includes one or more accelerometers measuring movement, vibration, and rotation in all axes.

In some embodiments, measuring device 400 includes one or more galvanic skin response (GSR) sensors between two or more contact points. In some embodiments, measuring device 100 includes one or more sensors for measuring electrical activity such as ECG, EMG and EEG.

Measuring device 100 may further include a power source connected to the two or more sensors. In some embodiments, power source has a measurable power capacity. In some embodiments, the sensors activation rules include activation intervals associated with the measured power capacity. For example, the activation frequency of at least one of the two or more sensors is reduced when the measured power capacity is reduced. In another example, the selection of a sensors to be activated is in accordance with the measured available power capacity. In some embodiments, power source is a battery and measured power source is equivalent to the level of battery charging level.

According to some embodiments, switching circuit 416 is configured to generate activation signals to one or more sensors of the two or more sensors. In some embodiments, switching circuit 416 is activated by a source outside measuring device 400.

In some embodiments, output unit 422 is adapted to output clinical parameters values of at least 1 minute of clinical parameters measurements. In some embodiments, output unit 422 is adapted to output clinical parameters values of at least 5 minutes of clinical parameters measurements. In some embodiments, output unit 422 is adapted to output clinical parameters values of at least 10 minutes of clinical parameters measurements. Such clinical parameters values output cycle may allow continuous measuring and monitoring of clinical parameters.

Measuring device 400, according to the invention, can be used for continuous monitoring of one or more clinical parameters for a long period of time. The exemplary operational parameters provided elsewhere herein are for demonstrating the continuous monitoring. Some examples of the clinical parameter are: systolic blood pressure, diastolic blood pressure, mean arterial pressure, pulse pressure, stroke volume, cardiac output, cardiac index, systemic vascular resistance, blood oxygen saturation, tissue oxygen saturation, respiratory rate, breathing volume, Heart rate, Heart rate variability (HRV), cardiac arrhythmia, level of sweat, movements, gait, calories consumption, body temperature, Hemoglobin level, glucose/sugar level, sleep quality, lactate, bilirubin level, fat level and a combination thereof.

According to some embodiments, measuring device 400 further includes a communication circuit adapted to communicate with one or more other devices (e.g., devices having similar functionality to the measuring device 400, or other connectable devices). In some embodiments, communication circuit 422 has a receiver circuit for receiving data from the one or more other measuring devices. In some embodiments, communication circuit 422 has a transmitter for transmitting data to the one or more other measuring devices. Such data may be received and/or sent over a wireless medium, protocol or technique. According to some embodiments, electrical circuitry 410 has a calibration functionality adapted to receive data from the one or more other measuring devices, and to output calibration data. In some embodiments, the calibration functionality receives as an input calibration data of the one or more other measuring devices and processes the calibration data.

According to some embodiments, electrical circuitry 410 controls the operation of measuring device 400 (e.g., sensors switching circuit 416, processor 414, and output unit 420). In some embodiments, electrical circuitry 410 is configured to trigger measuring of clinical parameters during specific conditions evaluated based on signals received from the two or more sensors. In some embodiments, electrical circuitry 410 is configured to trigger measuring of clinical parameters during specific conditions evaluated based on signals received from one or more cardiac or cardiovascular sensors 404. In some embodiments, electrical circuitry 410 is configured to trigger measuring of clinical parameters during specific conditions evaluated based on signals received from one or more additional sensors 408. Some examples of such conditions are:

Normal cardiac sinus rate is detected by an electrogram sensor (e.g., ECG); cardiac arrhythmia is detected by an electrogram sensor (e.g., ECG); Epileptic condition is detected by an electrogram sensor (e.g., EEG); Sleep stages like REM, non-REM, deep sleep, are detected by an electrogram sensor (e.g., EOG or EMG); and sweating periods as detected by an electrogram sensor that monitors changes in the electric activity of the tissue.

HR below or above set threshold, HRV below or above set threshold (e.g., lower than 40-60), BP values above or below set threshold (e.g., higher than 80-140). In some embodiments, measuring device 400 includes a setting functionality for setting one or more of above thresholds.

According to some embodiments, the measuring device used for measuring process 100 has stored threshold values or function for diastolic and systolic BP values, that depend on the BP baseline values and a set of programmable deviation. In some embodiments, deviation can be for example BP increase by 5%, 10%, 20% and higher.

The terms “comprise”, “comprising”, “includes”, “including”, “has”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure. As used herein, the singular forms “a”, “an” and “the” include plural references unless the context clearly dictates otherwise.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range. Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number is used herein interchangeably and is meant to include the first and second indicated numbers and all the fractional and integral numbers therebetween. Unless otherwise indicated, numbers used herein and any number ranges based thereon are approximations within the accuracy of reasonable measurement and rounding errors as understood by persons skilled in the art

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments unless the embodiment is inoperative without those elements. Although the invention has been described in conjunction with specific embodiments thereof, it is evident that many alternatives, modifications, and variations will be apparent to those skilled in the art. Accordingly, it is intended to embrace all such alternatives, modifications and variations.

What is claimed is:

1. A method for evaluating a normal range of a blood pressure, the method comprising:
executing a function,
wherein the function receiving as input measured values of one or more physiological parameters of a person,
wherein the function receiving as input measured values of one or more environmental parameters, and
wherein the function outputs a normal range of blood pressure values for the person according to the measured values of one or more physiological and/or environmental parameters.

2. The method of claim 1, further comprising:
measuring reference blood pressure values at several initial time points from the person;
measuring additional reference values of one or more additional physiological or environmental parameters of the patient during the same several initial time points, wherein the one or more additional or environmental physiological parameters are not blood pressure; and
generating the function based on the measured reference blood pressure values and the reference values of the one or more additional physiological or environmental parameters.

3. The method of claim 1, wherein the normal range is defined between a minimal normal BP value and a maximal normal BP value, wherein minimal normal BP value and the maximal normal BP value depend on the one or more additional physiological or environmental parameters.

4. The method of claim 1, further comprising measuring test blood pressure value.

5. The method of claim 4, further comprising measuring value of the one or more additional physiological or environmental parameters at the same time of measuring the test blood pressure value.

6. The method of claim 4, further comprising determining whether the measured test blood pressure value is within the normal range of the function pending on the measured additional parameter.

7. The method of claim 4, further comprising outputting a notice or an alert in case the measured test blood pressure value of the patient differs from the normal range of values.

8. The method of claim 4, further comprising outputting a notice or an alert in case the measured test blood pressure value differs from the normal range by a value higher than a predefined threshold.

9. The method of claim 4, wherein the test blood pressure value is measured in a continuous manner by a measuring device coupled to a patient.

10. The method of claim 1, further comprising updating the normal range according to one or more environmental conditions or activities.

11. The method of claim 10, wherein the one or more environmental conditions are selected from a group comprising hospitalization, patient's velocity relative to the ground, patient's location, patient's body posture, ambient conditions like temperature, humidity, height above sea level and a combination thereof.

12. The method of claim 1, further comprising updating the function for blood pressure values using additional blood pressure values measured after initial several time points.

13. The method of claim 1, wherein the one or more additional physiological or environmental parameters comprise at least one of a group comprising heart rate, heart rate variability, breathing rate, blood oxygen saturation, and a combination thereof.

14. The method of claim 2, wherein the reference blood pressure values comprise diastolic blood pressure values and systolic blood pressure values.

15. The method of claim 1, wherein measuring reference values of two or more additional physiological or environmental parameters other than the blood pressure during the initial several times points; and
generating the function based on the two or more additional physiological or environmental parameters.

16. The method of claim 2, wherein the function is a lookup table associating the reference blood pressure values and the one or more additional physiological or environmental parameters values.

17. The method of claim 2, wherein measuring the reference blood pressure values is during a predefined baseline time period.

18. The method of claim 1, wherein generating the function is by one or more techniques selected from a group comprising: polynomic fit, interpolation and extrapolation.

19. The method of claim 5, further comprising calculating an adjusted BP value using the measured BP value and the reference values of the one or more additional physiological or environmental parameters.

20. The method of claim 1, wherein measuring test blood pressure value in a periodical manner.

21. The method of claim 1, wherein the reference blood pressure values comprise at least one of systolic or diastolic BP values.

22. The method of claim 4, wherein the test blood pressure value comprises at least one of systolic or diastolic BP values.

23. The method of claim 1, wherein the personalized BP function is generated according to rules that consider objective information of the person.

24. The method of claim 1, wherein the personalized BP function is generated according to additional reference values.

25. The method of claim 2, wherein the normal range is personalized.

26. The method of claim 25, wherein the normal range is generated according to rules that consider objective information of the person.

27. The method of claim 25, wherein the normal range is generated according to additional reference values of the person.
